(19) United States
(12) Patent Application Publication
(10) Pub. No.: US 2012/0040378 A1
(43) Pub. Date: Feb. 16, 2012

(54) Meprobromate Immunoassay

(75) Inventors: Elouard Benchikh, Crumlin (GB); Ivan McConnell, Crumlin (GB); Peter Fitzgerald, Crumlin (GB); Philip Lowry, Crumlin (GB)

(73) Assignee: RANDOX LABORATORIES LIMITED, Crumlin (GB)

(21) Appl. No.: 13/205,833

(22) Filed: Aug. 9, 2011

(30) Foreign Application Priority Data
Aug. 10, 2010 (EP) 10172415.1

(51) Int. Cl. G01N 33/53; C07K 14/765; C07K 14/00; C07K 14/435; C07K 16/44; C07K 14/76; C07K 14/77

(52) U.S. Cl. 435/7.92; 530/300; 530/363; 530/367; 530/386; 530/400; 530/389.8; 436/501

(57) ABSTRACT

Carisoprodol is a centrally-acting prescription drug of known abuse. Upon ingestion it is rapidly metabolised to meprobamate, also a prescription drug with abuse potential. Current immunoassays are specific for carisoprodol and therefore have a short window of detection and, furthermore, are ineffective at detecting meprobamate. The current invention, underpinned by an antibody specific for meprobamate, overcomes these deficiencies.

### FIELD OF THE INVENTION

The invention relates to analytical methods for drug detection, and describes immunogens, antibodies, methods and kits for the detection and/or determination of meprobamate and carisoprodol.

### BACKGROUND TO THE INVENTION

Carisoprodol and meprobamate are centrally-acting drugs prescribed as muscle-relaxants and sedatives. Both have been implicated with abuse, drug overdose and driving impairment, especially meprobamate which possesses a Schedule IV rating under the Controlled Substances Act in the US. Meprobamate is also the principal metabolite of carisoprodol and is rapidly formed upon carisoprodol ingestion. Carisoprodol has a serum half-life of under 2 hours with less than 1% of a single orally administered carisoprodol tablet (350 mg) excreted unchanged in urine (Olsen et al 1994; Baselt R. C. 2008). Meprobamate has a plasma half-life of 6-16 hours and approximately 10-12% is excreted unchanged in the urine (Meprobamate: AHFS Drug Information).

There are three commercially available immunoassay test kits for carisoprodol. However, due to the rapid metabolism of carisoprodol these kits are unsuitable for long-term detection of the drug in individuals as it is not detectable in serum beyond 12 hours (Olsen et al 1994). There are no known immunoassays targeting meprobamate. Tests for meprobamate utilise gas chromatography linked to mass spectrometry (GC-MS) and gas chromatography (GC) which are expensive, require specialist staff and necessitate sample pre-treatment steps (Daval et al 2006; Gaillard et al 1997). There is a need for a cheap, practical and sensitive meprobamate-specific assay enabling an extended detection window for carisoprodol and meprobamate ingestion. The current invention describes such an assay.

### BIBLIOGRAPHY

Baselt R. C. (2008). In Disposition of Toxic Drugs and Chemicals in Man. 8^{th} edition, Biomedical Publications, Foster City, Calif., 2008, pp. 920-2.
Daval et al. (2006). J. Anal. Toxicol. 30: 302-5.
Gaillard Y et al. (1997). Forensic Sci Int., 86: 173-80.
Olsen H. et al. (1994). Ther. Drug Monit., 16: 337-340.
Robertson M. D. and Marinetti L. J. (2003). Forensic Sci. Rev., 15: 1-10.

### SUMMARY OF THE INVENTION

Described herein is the first known immunoassay for the detection and/or determination of meprobamate and carisoprodol. The immunoassay format of the present invention overcomes resource and practical limitations associated with previously described analytical methods used to detect meprobamate and/or carisoprodol.

### DESCRIPTION OF FIGURES

FIG. 1 Structures of meprobamate and carisoprodol.

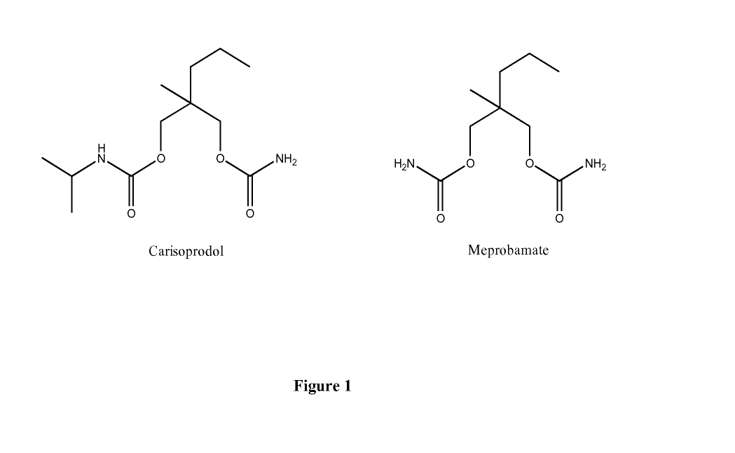

FIG. 2 Preparation of a carisoprodol hapten (Hapten-A).

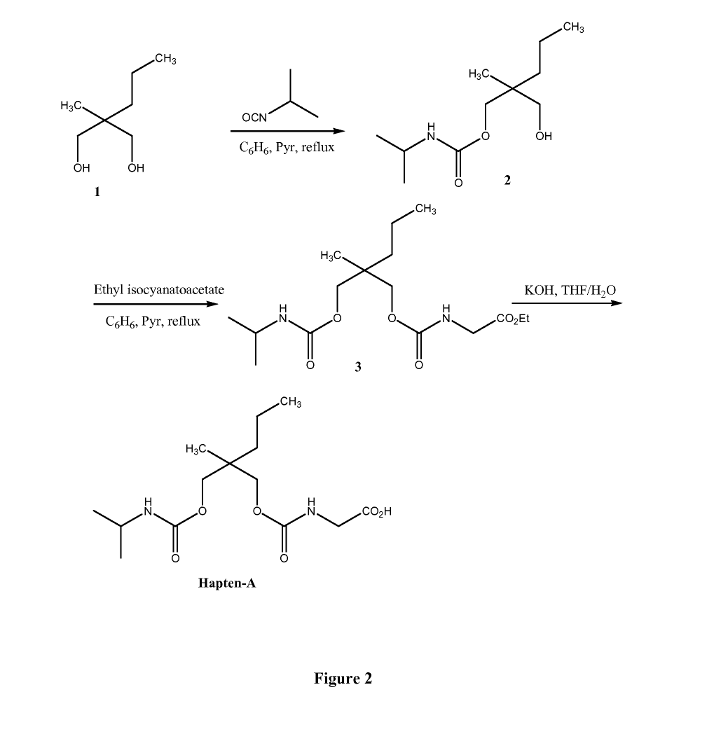

FIG. 3 Preparation of a meprobamate hapten (Hapten-B).

FIG. 4 Preparation of Immunogen 4 by conjugation of Hapten-B to BTG using N,N-dicyclohexylcarbodiimide (DCC) and N-hydroxysuccinimide (NHS).

### DETAILED DESCRIPTION OF THE INVENTION

In a first aspect, the present invention provides immunogens comprising meprobamate coupled by way of a crosslinker through one of the nitrogen atoms to an antigenicity-conferring carrier material (accm). The immunogens of the present invention correspond to structures of the formula of Structure I

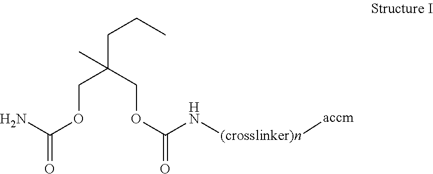

The immunogens are formed from the corresponding haptens (examples of which are to be found in the Methods section). Addition of a crosslinker to a nitrogen atom of meprobamate produces the (R,S)-hapten due to formation of an asymmetric centre. For the purpose of the invention the racemic hapten is used to prepare the described immunogens. Alternatively, either of the individual haptenic stereoisomers (i.e. ‘R’ or ‘S’) could be used for immunogen formation. The immunogens described in the invention are synthesised from the corresponding hapten, by preferably attaching a crosslinker to the meprobamate to form the hapten, and subsequently directly attaching the hapten to an accm. A crosslinker, well known in the art, prior to attachment to meprobamate is normally a bifunctionalised molecule that bridges two discrete molecules (in this case meprobamate and the accm). Optionally, the immunogen lacks a crosslinking group and meprobamate is bound directly to a suitable accm.

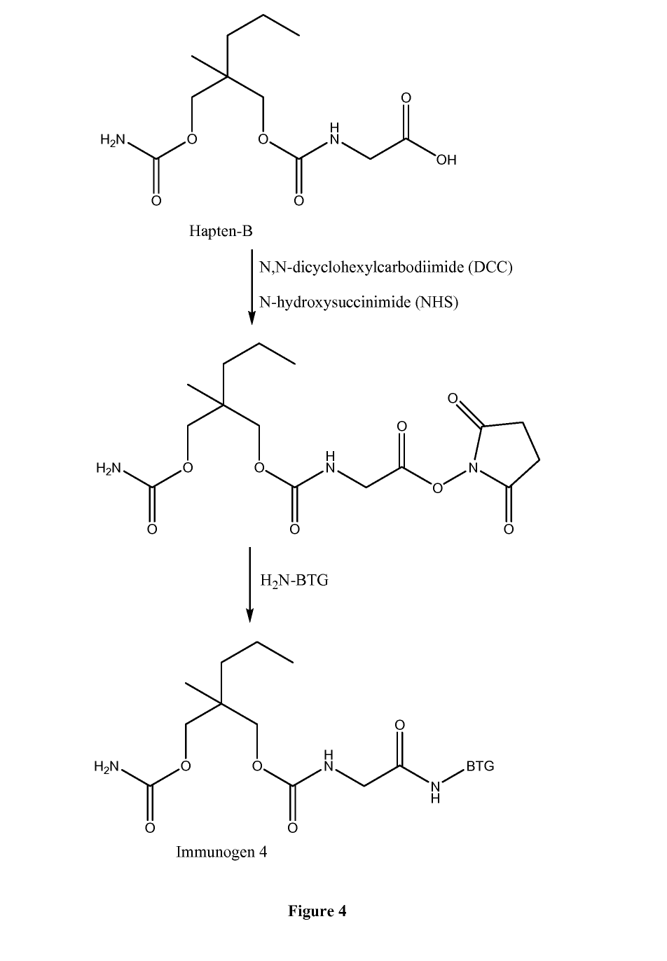

The crosslinker is —Y—Z—, where Y is a C₁-C₁₀, preferably a C₁-C₆, more preferably a C₁-C₃, substituted or unsubstituted straight chain alkylene moiety, or arylene moiety; Z (before conjugation with the accm) is selected from a carboxy, a dithiopyridyl, a maleimide, an amino, a hydroxyl, a thiol, a thioester or an aldehyde moiety, most preferably a carboxy moiety. Optionally, Y is a C₂ substituted or unsubstituted straight chain alkylene moiety, e.g. methylene.

Where Z, before conjugation to the accm is a carboxylic acid (COOH), the oxygen of the hydroxyl group combines first with DCC and then NHS to form an ester with a powerful leaving group. Nucleophilic attack on the carbonyl group (C═O) of the ester functionality by a free amine group on the accm results in an amide bond and formation of the desired immunogen. This reaction mechanism is illustrated, in brief, in FIG. 4 with respect to Immunogen 4, described hereinafter. Conjugate formation follows a similar mechanism using EDC and sulfo-NHS. The skilled reader is referred to Bioconjugate Techniques, G. Hermanson ed., Academic Press, 1996, 785 pp for details of the interaction between the Z reactive group and the accm. Most advantageously, Z, before conjugation with an accm to produce an immunogen, is a carboxy (COOH) moiety.

In a preferred embodiment, before conjugation to the accm, the crosslinker —Y—Z— is —CH₂—COOH.

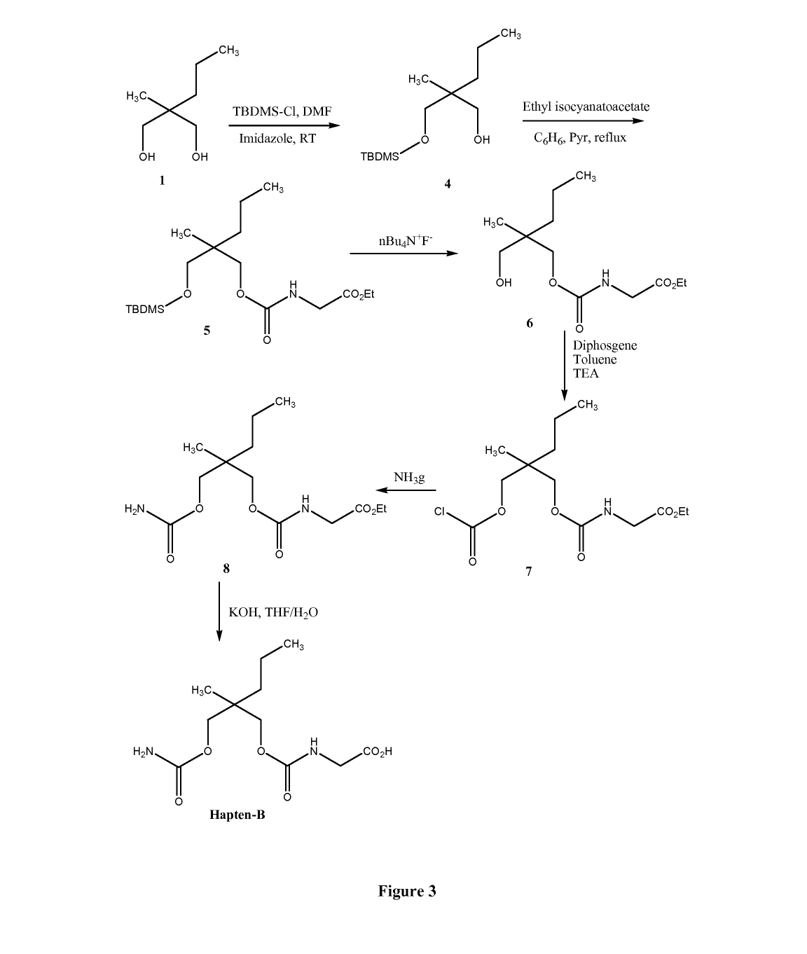

The immunogens are prepared by coupling to a modified or non-modified accm. Preferably the accm contains poly(amino acid) segments and includes proteins, protein fragments, glycoproteins, synthetic polypeptides or semi-synthetic polypeptides. Illustrative examples of useful antigenicity-conferring carrier materials are bovine serum albumin (BSA), egg ovalbumin, bovine gamma globulin, bovine thyroglobulin (BTG), keyhole limpet haemocyanin (KLH) etc. Alternatively, synthetic poly(amino acids) having a sufficient number of available amino groups, such as lysine, may be employed, as may other synthetic or natural polymeric materials bearing reactive functional groups. The immunogens obtained are then administered to mammalian hosts to elicit production of specific antibodies, optionally polyclonal antibodies, which are then used to develop immunoassays for meprobamate and carisoprodol, employing labelled conjugates as detection reagents. Preferably, the immunogen is N-carboxymethylmeprobamate (Hapten-B FIG. 3) coupled to an accm, the accm optionally selected from bovine serum albumin (BSA) and bovine thyroglobulin (BTG). In a preferred embodiment, the immunogen is Hapten-B coupled to BTG.

In a still further aspect, the present invention concerns antibodies raised against the immunogens of the present invention, the antibodies being capable of binding with at least one structural epitope of meprobamate, carisoprodol and related molecules.

In a still further aspect, the present invention concerns antibodies having specificity for a structural epitope of meprobamate and having cross-reactivity for carisoprodol. Antibodies that are specific for a certain molecule implies that the antibodies bind preferably to that molecule compared to other molecules. This is commonly represented by the skilled person using the cross-reactivity concept, in which the antibody-specific molecule has 100% cross-reactivity to the antibody and all other molecules have a cross-reactivity of less than 100% to the antibody. The antibodies of the current invention are specific to an epitope of meprobamate and may have cross-reactivity to an epitope of carisoprodol. The cross-reactivity of the antibodies to carisoprodol is >10%, preferably >30%, most preferably >50% but is always less than the cross-reactivity to meprobamate. Optionally, the cross-reactivity of the antibodies to carisoprodol is >55%. Similarly, the meprobamate-specific antibodies of the invention can cross-react (less than 100% compared to meprobamate) with mebutamate, tybamate and similar N-monoalkylated analogues of meprobamate. In a still further aspect, the present invention comprises a method for detecting or determining meprobamate and/or carisoprodol in an in vitro sample, the method comprising contacting the sample with at least one conjugate, and with at least one antibody of the present invention; detecting or determining bound conjugate; and deducing from a calibration curve the presence of, or the amount of meprobamate and/or carisoprodol in the sample. Detecting implies the qualitative analysis of the presence or absence; determining means the quantitative analysis. As is known by the skilled man, for a quantitative competitive immunoassay incorporating an antibody which is able to bind to an epitope (whether the same or different) of more than one molecule, the determined value is derived from calibrator or standard equivalents. Within the context of the current invention calibrator/standard are synonymous unless otherwise stated. In order to derive quantitative values for the individual molecules meprobamate and carisoprodol, a two antibody immunoassay approach could be used, incorporating, for example, the antibodies of the invention and an additional antibody that binds either meprobamate or carisoprodol, but not both. Table 1 shows the cross-reactivity profile of an antibody specific to carisprodol produced by the inventors which when utilised with the antibody of the invention would enable individual quantification of both meprobamate and carisoprodol.

In a further aspect, the invention includes a kit for detecting meprobamate and/or carisoprodol, the kit including at least one antibody of the present invention and optionally including at least one conjugate. Alternatively, the kit is for determining meprobamate and/or carisoprodol, the kit including at least one antibody of the present invention and optionally including at least one conjugate. The kit may optionally include instructions for the use of said conjugates and said antibodies for determining the amount of each of meprobamate and/or carisoprodol in a sample. The sample can be any biological fluid but is preferably whole blood.

### Methods and Results

### General Procedure for MALDI-TOF Analysis of Immunogens.

In order to confirm that adequate conjugation to an antigenicity-conferring carrier material has been achieved, prior to immunisation, each immunogen is evaluated using matrix-assisted UV laser desorption/ionisation time-of-flight mass spectroscopy (MALDI-TOF MS). MALDI-TOF mass spectrometry was performed using a Voyager STR Biospectrometry Research Station laser-desorption mass spectrometer coupled with delayed extraction. An aliquot of each sample to be analysed was diluted in 0.1% aqueous trifluoroacetic acid (TFA) to create 1 mg/ml sample solutions. Aliquots (1 μl) were analysed using a matrix of sinapinic acid and bovine serum albumin (Fluka) was used as an external calibrant.

### Preparation of Antisera

In order to generate polyclonal antisera, the immunogen of the present invention is mixed with Freund's Adjuvant and the mixture is injected into a host animal, such as rabbit, sheep, mouse, guinea pig or horse. Further injections (boosts) are made and serum is sampled for evaluation of the antibody titre. When the optimal titre has been attained, the host animal is bled to yield a suitable volume of specific antiserum. The degree of antibody purification required depends on the intended application. For many purposes, there is no requirement for purification, however, in other cases, such as where the antibody is to be immobilised on a solid support, purification steps can be taken to remove undesired material and eliminate non-specific binding. The specific antibodies prepared in this invention are useful as reagents in immunoassays for the detection or determination of meprobamate and carisoprodol in biological fluids. The antibodies of the present invention are also capable of binding with N-monoalkylated analogues of meprobamate.

### Example 1

### Preparation of 2-methyl-2-hydroxymethylpentyl N-isopropylcarbamate 2

To a stirred solution of 2-methyl-2-propyl-1,3-propanediol 1 (19.83 g, 0.15 mol) in anhydrous toluene (250 ml) was added isopropyl isocyanate (16.21 ml, 0.165 mol). The mixture was heated at reflux for 5 h. The reaction mixture was allowed to cool to room temperature, water (100 ml) was added to the mixture and the two layers were separated. The aqueous layer was extracted with ethyl acetate (1×100 ml) and the combined organic layers were washed with water (100 ml), brine (100 ml), dried over sodium sulphate, filtered and concentrated in vacuo. The crude product (26.6 g) was purified by chromatography on silica gel (hexane/ethyl acetate: 1/1) to give 2-methyl-2-hydroxymethylpentyl N-isopropylcarbamate 2 (22.5 g, 69%) as a yellow oil.

### Example 2

### Preparation of 2-methyl-2-propyl-1,3-propanediol N-isopropyl-N-ethoxycarbonylmethyldicarbamate 3

To a stirred solution of 2 (21.731 g, 0.1 mol) in anhydrous toluene (300 ml) was added pyridine (10 ml) and ethyl isocyanatoacetate (13.5 ml, 0.12 mol) and the mixture was heated at reflux overnight. The solution was then cooled at room temperature, water was added (150 ml) and the two layers separated. The aqueous layer was extracted by ethyl acetate (2×100 ml) and the combined organic layers were washed with water (2×100 ml), brine (100 ml), dried over sodium sulphate, filtered and concentrated to dryness to give the pure compound 3 as a yellow oil (20.7 g, 59.7%).

### Example 3

### Preparation of 2-methyl-2-propyl-1,3-propanediol N-isopropyl-N-carboxymethyldicarbamate(N-{carboxymethyl)carisoprodol/Hapten-A)

Compound 3 (3.46 g, 0.01 mol) was dissolved in a mixture of tetrahydrofuran (THF) (100 ml) and water (100 ml). Potassium hydroxide (KOH) (4.37 g, 0.032 mol) was added and the mixture stirred overnight at room temperature. The THF was removed under reduced pressure and the aqueous solution acidified to pH 2 by HCl (2N). The white solid formed was filtered, washed with water and dried under vacuum overnight to give Hapten-A (2.9 g, 91%).

NMR¹³C (δ: ppm): 172.47, 158.20, 157.19, 68.79, 42.92, 41.96, 37.66, 36.85, 21.80, 18.17, 16.33 and 14.13

### Example 4

### Conjugation of Hapten-A to BSA (Immunogen 1)

To a solution Hapten-A (35.82 mg, 0.1128 mmol) in DMF (1.0 ml) was added N,N-dicyclohexylcarbodiimide (DCC) (25.35 mg, 0.123 mmol) and N-hydroxysuccinimide (14.13 mg, 0.123 mmol) and the mixture was stirred at room temperature overnight. The dicyclohexylurea formed was removed by filtration and the solution was added dropwise to a solution of BSA (150 mg, 2.3 mmol) in 50 mM sodium bicarbonate solution (10 ml, pH 8.5). The mixture was stirred overnight at 4° C. The solution was dialysed against 50 mM phosphate buffer pH 7.2 (3 changes) for 24 h at 4° C., and freeze-dried to give 141 mg of Immunogen 1. MALDI results showed 31.23 molecules of Hapten-A had been conjugated to one molecule of BSA.

### Example 5

### Conjugation of Hapten-A to BTG (Immunogen 2)

To a solution of Hapten-A (43.91 mg, 0.135 mmol) in DMF (1.0 ml) was added N,N-dicyclohexylcarbodiimide (DCC) (30.7 mg, 0.149 mmol) and N-hydroxysuccinimide (17.13 mg, 0.149 mmol) and the mixture stirred at room temperature overnight. The dicyclohexylurea formed was removed by filtration and the solution was added dropwise to a solution of BTG (150 mg, 2.25 υmol) in 50 mM sodium bicarbonate solution (10 ml, pH 8.5). The mixture was then stirred overnight at 4° C. The solution was then dialysed against 50 mM phosphate buffer pH 7.2 (3 changes) for 24 h at 4° C., and freeze-dried to give 145 mg of Immunogen 2.

### Example 6

### Conjugation of Hapten-A to HRP

EDC hydrochloride (10 mg) was dissolved in water (0.5 ml) and immediately added to a solution of Hapten-A (2 mg) in DMF (0.2 ml). After mixing, this solution was added dropwise to a solution of HRP (20 mg) in water (1 ml). Sulfo-NHS (5 mg) was added and the reaction mixture was incubated in the dark at room temperature overnight. Excess hapten was removed with double PD-10 columns (Pharmacia) in series, pre-equilibrated with PBS at pH 7.2. The hapten-HRP conjugate was then dialysed overnight against 10 L of PBS at pH 7.2 at 4° C.

### Example 7

### Preparation O-terbutyldimethylsilyl 2-methyl-2-propyl-1,3-propanediol 4

To a stirred solution of 2-methyl-2-propyl-1,3-propanediol 1 (19.83 g, 0.15 mol) in DMF (200 ml) at 0° C. was added imidazole (20.42 g, 0.3 mol) and TBDMS-CL (24.9 g, 0.165 mol) and the mixture was stirred at room temperature overnight. Water (300 ml) was added to the mixture and the solution was extracted with diethyl ether (2×300 ml). The combined diethyl ether layers were washed by water (1×200 ml), brine (1×100 ml), dried over sodium sulphate, filtered and concentrated to dryness. The crude product obtained was purified by flash chromatography on silica gel using 15% ethyl acetate/85% hexane to give O-terbutyldimethylsilyl 2-methyl-2-propyl-1,3-propanediol 4 (22.1 g, 60%) as a colourless oil.

### Example 8

### Preparation of 2-(O-terbutyldimethylsiloxymethyl)-2-methylpentyl N-ethoxycarbonylmethylcarbamate 5

To a stirred solution of 4 (11.0 g, 44.71 mmol) in anhydrous toluene (200 ml) was added pyridine (10 ml) and ethyl isocyanatoacetate (6.6 ml, 58.12 mmol) and the mixture heated at reflux overnight. The solution was cooled at room temperature, water added (150 ml) and the two layers separated. The aqueous layer was extracted with ethyl acetate (2×100 ml) and the combined organic layers were washed with water (2×100 ml), brine (100 ml), dried over sodium sulphate, filtered and concentrated to dryness to give 2-(O-terbutyldimethylsiloxymethyl)-2-methylpentyl N-ethoxycarbonylmethylcarbamate 5 (17.27 g) as a milky liquid.

### Example 9

### Preparation of 2-hydroxymethyl-2-methylpentyl N-ethoxycarbonylmethylcarbamate 6

To s stirred solution of 5 (17.0 g, 45.33 mmol) in THF (300 ml) at 0° C. was added tetrabutylammonium fluoride (17.16 g, 54.4 mmol) and the mixture stirred at room temperature for 4 h. The solvent was removed in vacuo and the residue purified by flash chromatography on silica gel using 30% ethyl acetate/70% hexane to give the pure compound 6 as a clear viscous oil (9.2 g, 77.7%).

### Example 10

### Preparation on 2-methyl-2-propyl-1,3-propanediol N-ethoxycarbonylmethyldicarbamate 8

To a stirred solution of compound 6 (9.0 g, 34.5 mmol) in anhydrous toluene (150 ml) and TEA (5.2 ml) at 0° C. was added diphosgene (2.1 ml, 17.25 mmol). The mixture was stirred at room temperature for 1 h. Dichloromethane (150 ml) was added and the mixture cooled to −78° C. and ammonia gas bubbled through for 10 min. The mixture was allowed to warm to room temperature and the solvent removed in vacuo. The residue obtained was triturated with ethyl acetate and the mixture filtered. The filtrate was concentrated to dryness and triturated with hexane/diethyl ether (50/50) to give 2-methyl-2-propyl-1,3-propanediol N-ethoxycarbonylmethyldicarbamate 8 as a white solid (8.3 g, 79%).

### Example 11

### Preparation of 2-methyl-2-propyl-1,3-propanediol N-carboxymethyldicarbamate(N-(carboxymethyl)meprobamate/Hapten-B)

Compound 8 (4.8 g, 15.8 mmol) was dissolved in a mixture of tetrahydrofuran (THF) (100 ml) and water (100 ml). Potassium hydroxide (KOH) (5.3 g, 47.5 mmol) was added and the mixture stirred overnight at room temperature. The THF was removed under reduced pressure, the aqueous solution acidified to pH 3-4 with HCl (2N) and the solution extracted with ethyl acetate (2×10 ml). The combined organic extracts where washed with water (1×100 ml), brine (1×100 ml), dried over sodium sulphate, filtered and concentrated to dryness to give Hapten-B as a clear gum (3.5 g, 80%).

NMR¹³C (δ ppm): 174.65, 160.93, 159.92, 70.26, 69.90, 43.74, 39.13, 38.29, 19.72, 17.84 and 15.85.

MS: [M-H]⁺: 275.1241.

### Example 12

### Conjugation of Hapten-B to BSA (Immunogen 3)

To a solution Hapten-B (31.05 mg, 0.1125 mmol) in DMF (1.0 ml) was added N,N-dicyclohexylcarbodiimide (DCC) (25.35 mg, 0.123 mmol) and N-hydroxysuccinimide (14.13 mg, 0.123 mmol) and the mixture stirred at room temperature overnight. The dicyclohexylurea formed was removed by filtration and the solution was added drop-wise to a solution of BSA (150 mg, 2.3 mmol) in 50 mM sodium bicarbonate solution (pH 8.5) (10 ml). The mixture was stirred overnight at 4° C. The solution was dialysed against 50 mM phosphate buffer pH 7.2 (3 changes) for 24 h at 4° C., and freeze-dried to give 135 mg of Immunogen 3. MALDI results showed 27.04 molecules of Hapten-B had been conjugated to one molecule of BSA.

### Example 13

### Conjugation of Hapten-B to BTG (Immunogen 4)

To a solution of Hapten-B (37.29 mg, 0.135 mmol) in DMF (1.0 ml) was added N,N-dicyclohexylcarbodiimide (DCC) (30.7 mg, 0.149 mmol) and N-hydroxysuccinimide (17.13 mg, 0.149 mmol) and the mixture stirred at room temperature overnight. The dicyclohexylurea formed was removed by filtration and the solution added dropwise to a solution of BTG (150 mg, 2.25 υmol) in 50 mM sodium bicarbonate solution (10 ml, pH 8.5). The mixture was stirred overnight at 4° C. then dialysed against 50 mM phosphate buffer pH 7.2 (3 changes) for 24 h at 4° C., and freeze-dried to give 140 mg of Immunogen 4. This reaction mechanism is illustrated, in brief, in FIG. 4.

### Example 14

### Conjugation of Hapten-B to HRP

EDC hydrochloride (10 mg) was dissolved in water (0.5 ml) and added to a solution of Hapten-B (2 mg) in DMF (0.2 ml). After mixing, the solution was added dropwise to a solution of HRP (20 mg) in water (1 ml). Sulfo-NHS (5 mg) was added and the reaction mixture was incubated in the dark at room temperature overnight. Excess hapten was removed with double PD-10 columns (Pharmacia) in series, pre-equilibrated with PBS at pH 7.2. The hapten-HRP conjugate was then dialysed overnight against 10 L of PBS at pH 7.2 at 4° C.

### Example 15

### Preparation of Antibodies to Immunogen 4

An aqueous solution of Immunogen 4 was formulated with Freund's Complete Adjuvant (FCA) to form an emulsion consisting of 2 mg/ml immunogen in 50% (v/v) FCA. Three sheep were immunised with this emulsion (1° immunisation), 0.25 ml being intramuscularly injected at each of four sites in the rump of each animal. Subsequent immunizations (boosts) contained 1 mg/ml immunogen. All boosts were emulsified in 50% (v/v) Freund's Incomplete Adjuvant (FIA) and were administered in the same manner as the 1° immunisation, at monthly intervals for 1 year. Blood sampling took place 7 to 14 days after each boost. Each sample was processed to produce antiserum, which was further purified by caprylic acid and ammonium sulfate precipitation to yield an immunoglobulin (Ig) fraction. The Ig fraction was evaluated by competitive ELISA microtiter plate assay, as described in Example 16.

### Example 16

### Development of ELISAs for Meprobamate and Carisprodol

The wells of an enhanced binding 96 well polystyrene microtiter plate were coated with a mixture of the Ig fractions of the antisera raised to Immunogen 4, diluted in 10 mM Tris, pH8.5 (125 μl/well). The appropriate antibody coating dilution was determined using standard ELISA checkerboard techniques. The plate was incubated for 2 hours at 37° C., washed 4 times with Tris buffered saline containing Tween 20 (TBST) and tapped dry. Standard solutions of meprobamate, carisoprodol and mebutamate were prepared in TBST at 0, 0.1, 1.0, 5, 10, 25, 50 and 100 ng/ml, and 50 μl of each was added to the appropriate wells.

The conjugate prepared in Example 14 diluted in Tris buffer (pH 7.2) containing EDTA, D-mannitol, sucrose, thimerosal and BSA, was added to each of the wells. The appropriate dilution of conjugate was also determined using standard ELISA checkerboard techniques. The plate was incubated at 25° C. for 1 hour. Excess unbound conjugate was removed by washing 6 times over a 10 minute period with TBST. 125 μl of tetramethylbenzidine (TMB) substrate solution was added to each well of the plate that was then incubated for 20 minutes in the dark at room temperature. The reaction was terminated by addition of 125 μl 0.2M H₂SO₄ to each well. The absorbance was then measured at 450 nm using a microtiter plate reader.

### Results

The calculated IC₅₀ and CR values for meprobamate and carisoprodol were 8.64 ng/ml and 100% and 15.15 ng/ml and 57%, respectively. The antibody displayed a cross-reactivity of 7% to the minor prescription drug mebutamate. Tybamate, of similar structure to carisoprodol, is expected to display cross-reactivity to the antibody, as would other N-monoalkylated meprobamate derivatives in which the alkyl chain is of short to medium length (approximately C₁-C₁₀). Table 1 compares the IC₅₀ and CR values of commercially available immunoassays and the immunoassay of the current invention. As can be seen, the antibody of the invention enables an immunoassay that is uniquely specific for meprobamate.

TABLE 1
Specificity and sensitivity of carisoprodol-specific
antibody raised from Immunogen 2

| Antibody property | Value |
|---|---|
| IC₅₀ carisoprodol (ng/ml) | 15 |
| IC₅₀ meprobamate (ng/ml) | 8 |
| % CR carisoprodol | 57 |
| % CR meprobamate | 100 |

TABLE 2
Specificity and sensitivity comparisons of
meprobamate and carisoprodol immunoassays

| Antibody property | Randox | Immunalysis* | Neogen** | IDS |
|---|---|---|---|---|
| IC₅₀ carisoprodol | 15 | 25 | nk | 0.6 |
| (ng/ml) | | | | |
| IC₅₀ meprobamate | 8 | 132 | nk | >6000 |
| (ng/ml) | | | | |
| % CR carisoprodol | 57 | 100 | nk | 100 |
| % CR meprobamate | 100 | 19 | nk | <0.01 |

IC₅₀ = standard concentration which produces 50% B/B₀;
% CR = percentage cross-reactivity based on 100% specificity to meprobamate (B = UV absorbance at 450 nm at x ng/ml calibrator concentration; B₀ = absorbance at 450 nm at 0 ng/ml calibrator concentration).
*Data from Robertson and Marinetti, 2003
**described as a carisoprodol immunoassay;
nk = not known

1. An immunogen of the structure 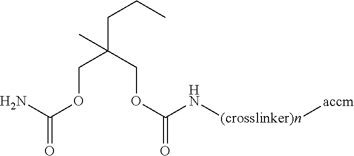
where n=0 or 1, the accm is an antigenicity-conferring carrier material and, where, when n=1, the crosslinker links a nitrogen atom of meprobamate to the accm.

2. The immunogen of claim 1 where n=1, and the crosslinker is —Y—Z—, where Y is a C₁-C₁₀, substituted or unsubstituted straight chain alkylene moiety, or arylene moiety; Z (before conjugation to the accm) is selected from a carboxy, a dithiopyridyl, a maleimide, an amino, a hydroxyl, a thiol, a thioester or an aldehyde moiety.

3. The immunogen of claim 2 where Y is methylene and Z (before conjugation to the accm) is a carboxy group.

4. An antibody raised against an immunogen of claim 1 that is specific for a structural epitope of meprobamate.

5. The antibody of claim 4 characterised by having cross-reactivity to carisoprodol.

6. The antibody of claim 5 which has a cross-reactivity to carisoprodol that is greater than 10%.

7. The antibody of claim 5 which has a cross-reactivity to carisoprodol that is greater than 30%.

8. The antibody of claim 5 which has a cross-reactivity to carisoprodol that is greater than 50%.

9. A method of detecting or determining of meprobamate and/or carisoprodol in an in vitro sample taken from an individual, the method comprising contacting the sample with a conjugate, and an antibody of claim 4, detecting the bound conjugate, and deducing from calibration value(s) the presence of or the calibrator-equivalent amount of meprobamate and/or carisoprodol.

10. A kit for detecting or determining meprobamate and/or carisoprodol, the kit comprising an antibody of claim 4.

11. The kit of claim 10 that also comprises at least one conjugate and/or at least one calibrator.
